(19) United States
(12) Patent Application Publication
(10) Pub. No.: US 2012/0142949 A1
(43) Pub. Date: Jun. 7, 2012

(54) PROCESS FOR PREPARING PREGABALIN

(75) Inventor: B. S. Pradhan, Hyderabad (IN)

(73) Assignee: HELVETICA INDUSTRIES (P) LIMITED, New Delhi (IN)

(21) Appl. No.: 13/388,745

(22) PCT Filed: Aug. 2, 2010
(86) PCT No.: PCT/IN2010/000510

§ 371 Date: Feb. 3, 2012

(30) Foreign Application Priority Data
Aug. 3, 2009 (IN) 1614/DEL/2009
Nov. 5, 2009 (IN) 2283/DEL/2009
May 26, 2010 (IN) 1221/DEL/2010

(51) Int. Cl. C07C 227/18; C07D 493/04

(52) U.S. Cl. 549/435; 562/575

(57) ABSTRACT

The invention relates to a process for preparing a compound of formula (I):

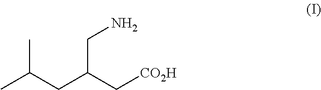

wherein said process comprises hydrogenation of a compound of formula (II);

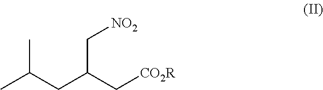

under alkaline conditions, wherein R represents hydrogen or a labile group capable of being converted to hydrogen. The invention also relates to intermediates used in said process, to the use of said intermediates in the preparation of pregabalin and to a process for resolving racemic compounds of formula (I).

### FIELD OF THE INVENTION

The invention relates to a process for preparing pregabalin, to intermediates used in said process, to the use of said intermediates in the preparation of pregabalin and to a process for resolving racemic compounds of formula (I).

### BACKGROUND OF THE INVENTION

Pregabalin is an anticonvulsant drug used for neuropathic pain and as an adjunct therapy for partial seizures with or without secondary generalization in adults. Pregabalin has also been found effective for generalized anxiety disorder and was approved in 2007 for this use in the European Union. It was designed as a more potent successor to gabapentin.

Pregabalin is known chemically as (S)-3-(aminomethyl)-5-methylhexanoic acid and has been developed for use in the treatment of epilepsy, pain, anxiety and social phobia. Pregabalin is an analog to 4-aminobutyric acid (GABA), a neurotransmitter that is thought to play a major inhibitory role in the central nervous system (CNS).

Recent studies have shown that pregabalin is effective at treating chronic pain in disorders such as fibromyalgia and spinal cord injury. In June 2007, pregabalin became the first medication approved by the U.S. Food and Drug Administration specifically for the treatment of fibromyalgia.

### SUMMARY OF THE INVENTION

According to a first aspect of the invention there is provided a process for preparing a compound of formula (I):

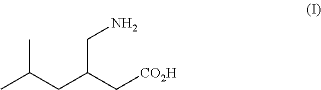

wherein said process comprises hydrogenation of a compound of formula (II)

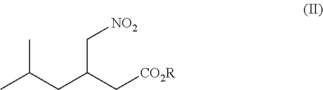

under alkaline conditions, wherein R represents hydrogen or a labile group capable of being converted to hydrogen.

According to a second aspect of the invention there is provided a process for preparing a compound of formula (I):

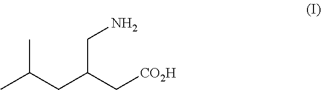

wherein said process comprises hydrolysis of a compound of formula (II)

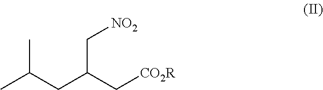

followed by hydrogenation under alkaline conditions, wherein R represents a labile group capable of being converted to hydrogen.

According to a further aspect of the invention there is provided intermediates (X), (II)b and (II)c.

According to a further aspect of the invention there is provided intermediates (X), (II)b and (II)c for use in the preparation of pregabalin.

### DETAILED DESCRIPTION OF THE INVENTION

According to a first aspect of the invention there is provided a process for preparing a compound of formula (I):

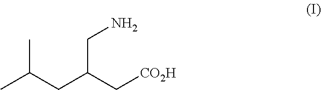

wherein said process comprises hydrogenation of a compound of formula (II)

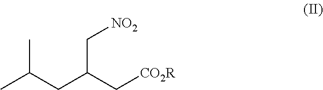

under alkaline conditions, wherein R represents hydrogen or a labile group capable of being converted to hydrogen.

The process described herein for the preparation of pregabalin and intermediates used therein provide a number of advantages. For example, the process is simple, efficient, and easy to operate as well as providing a good yield.

In a particular embodiment which may be mentioned, R represents a labile group capable of being converted to hydrogen. It will be appreciated that when R represents a labile group capable of being converted to hydrogen, said hydrolysis reaction may either be conducted prior to or subsequent to the hydrogenation reaction of the invention. In one embodiment, said hydrolysis reaction is conducted prior to the hydrogenation reaction of the invention. The significant advantage of performing the hydrolysis reaction prior to the hydrogenation reaction is that hydrolysis typically requires alkaline reagents such as potassium hydroxide. Such alkaline conditions have beneficially been shown by the inventors of the present invention to be optimal for the hydrogenation reaction required to prepare preqabalin

Therefore, the hydrolysis and hydrogenation reactions can be performed sequentially without a separate isolation step. Such a “one-pot” process is advantageous because compounds of formula (I) wherein R represents hydrogen (i.e. acidic compounds) are unstable and have been found to be unsuitable for hydrogenation via conventional reactions'in the art. The process of the invention therefore provides a greatly simplified preparation of pregabalin in only three steps.

Thus, according to a second aspect of the invention there is provided a process for preparing a compound of formula (I):

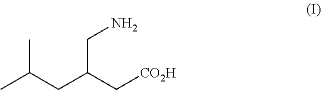

wherein said process comprises hydrolysis of a compound of formula (II)

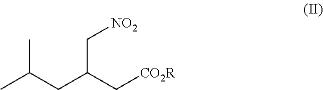

followed by hydrogenation under alkaline conditions, wherein R represents a labile group capable of being converted to hydrogen.

References herein to “alkaline conditions” refer to conducting the hydrogenation reaction under conditions which have a pH of greater than 7. In one embodiment, the alkaline conditions are created by addition of a suitable base to the hydrogenation reaction. In a further embodiment, the alkaline conditions are created by addition of a suitable base prior to the hydrogenation reaction. In one embodiment, said base is potassium hydroxide.

In one embodiment, the hydrogenation reaction of the invention comprises hydrogenation in the presence of a suitable catalyst. In a further embodiment, said catalyst comprises a metal selected from nickel, platinum, palladium, rhodium and ruthenium. In a further embodiment, said catalyst comprises a metal selected from nickel, such as Raney nickel or Urushibara nickel. In a further embodiment, said catalyst comprises Raney nickel.

In one embodiment, the hydrogenation reaction of the invention comprises hydrogenation at a pressure of between 100 and 500 kPa, such as 345 kPa.

In one embodiment, R represents C₁₋₆ alkyl. Examples of C₁₋₆ alkyl groups include methyl, ethyl, n-propyl, isopropyl, n-butyl, isobutyl, sec-butyl, tert butyl, n-pentyl, isopentyl, neopentyl or hexyl and the like. In a further embodiment, R represents ethyl (—CH₂CH₃).

In an alternative embodiment, R represents a compound of formula R^{a}:

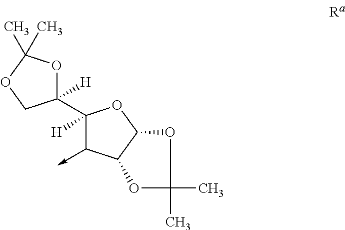

wherein [symbol] represents the point of attachment of R to the O atom of the CO₂ group.

In an alternative embodiment, R represents a compound of formula R^{b}:

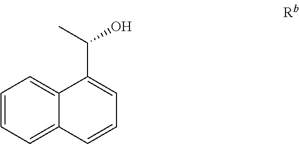

In an alternative embodiment, R represents a compound of formula R^{c}:

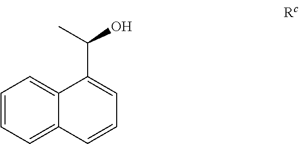

In one embodiment, the hydrolysis reaction comprises the addition of potassium hydroxide, such as potassium hydroxide cooled to 10-15° C. In a further embodiment, said potassium hydroxide is added at a temperature of between 0 and 5° C. In a yet further embodiment, said hydrolysis reaction is conducted at room temperature.

It will be appreciated that compounds of formula (I) may exist in a variety of differing optical configurations. For example, in one embodiment, the compound of formula (I) has the stereochemistry shown in the following compound of formula (I)^{a}:

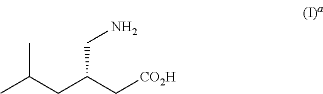

Compounds of formula (I)^{a} may be prepared from a racemic compound of formula (I) in accordance with known resolution procedures, such as those described in Hoekstra et al. (1997) Organic Process Research and Development 1, 26—by resolution with R(−)-mandelic acid.

In an alternative embodiment, the compound of formula (I) has the stereochemistry shown in the following compound of formula (I)^{b}:

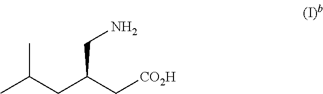

Compounds of formula (I)^{b} may be prepared from a racemic compound of formula (I) in accordance with known resolution procedures, such as those described in Hoekstra et al. (1997) Organic Process Research and Development 1, 26—by resolution with S(+)-mandelic acid.

In one embodiment, the resolution procedure comprises preparing the nitro compound of Example 9. This step of the resolution procedure typically comprises reacting nitromethane with an olefinic ester followed by addition of N,N-tetramethylguanidine to the mixture under an atmosphere of nitrogen. The reaction mixture is then subjected to distillation under reduced pressure to recover N,N-tetramethylguanidine. The residue after the removal of N,N-tetramethylguanidine contained primarily the nitro compound of Example 9.

In a further embodiment, the resolution procedure additionally comprises preparing racemic pregabalin from the nitro compound of Example 9 (for example in accordance with the procedure described in Example 10). Typically, the nitro compound of Example 9 is cooled and a cold solution potassium hydroxide is added.

Subsequently, after the completion of addition, the reaction mixture is allowed to warm to room temperature and stirred. Furthermore, methanol and ethanol are added to the reaction mixture to make it homogenous. Raney nickel is added to it and the reaction mixture is hydrogenated until the uptake of hydrogen stopped.

In a yet further embodiment, the resolution procedure additionally comprises preparing the resolved pregabalin from racemic pregabalin (for example in accordance with the procedure described in Example 11). Typically, the racemic pregabalin precipitated from the solution is filtered, washed with cold dichloromethane and resolved with (S)-mandelic acid.

Thus, according to a further aspect of the invention there is provided a process for preparing a resolved form of the compound of formula (I):

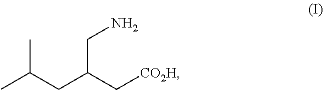

such as a compound of formula (I)^{b}

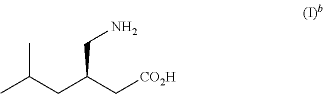

which comprises resolution of the racemic compound of formula (I) by S(+)-mandelic acid.

According to a further aspect of the invention there is provided a purified compound of formula (I)^{b}

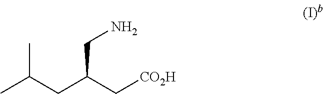

obtainable by the resolution process described hereinbefore.

Compounds of formula (II) wherein R represents ethyl may be prepared in accordance with Scheme 1:

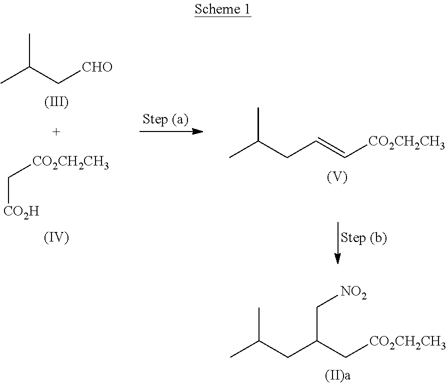

Step (a) typically comprises the reaction of a compound of formula (III) with a compound of formula (IV) in the presence of a suitable base, such as pyridine and piperidine at a suitable temperature, such as room temperature.

Step (b) typically comprises reacting a compound of formula (V) with nitromethane in the presence of a suitable catalyst, such as 1,8-diazabicyclo[5.4.0]undec-7-ene (DBU) at a suitable temperature, such as room temperature typically under an atmosphere of nitrogen.

Compounds of formula (II) wherein R represents R^{a} may be prepared in accordance with Scheme 2:

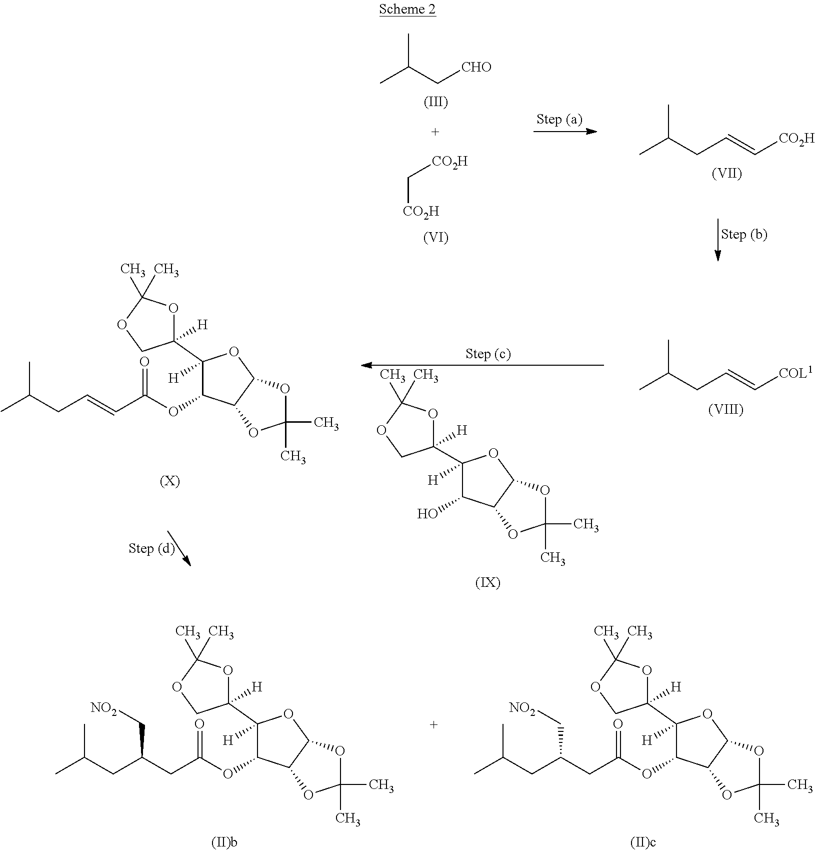

wherein L¹ represents a suitable leaving group, such as a halogen atom (e.g. chlorine).

Step (a) typically comprises the reaction of a compound of formula (III) with a compound of formula (VI) in an analogous manner to that described hereinbefore for Step (a) of Scheme 1.

When L¹ represents chlorine, step (b) typically comprises reaction of a compound of formula (VII) with thionyl chloride at a suitable temperature, such as 0-5° C.

Step (c) typically comprises reaction of a compound of formula (IX) with a suitable base, such as pyridine, followed by reaction with a compound of formula (VIII) followed by the addition of a suitable catalyst, such as N,N-dimethylaminopyridine (DMAP).

Step (d) comprises reaction of a compound of formula (X) in an analogous manner to that described hereinbefore for Step (b) of Scheme 1.

Compounds of formula (III), (IV), (VI) and (IX) are either known or may be prepared in accordance with known procedures.

In a particular embodiment which may be mentioned, R represents hydrogen. Thus, according to a further aspect of the invention there is provided a process for preparing a compound of formula (I):

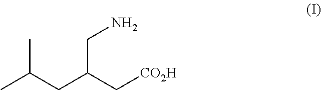

wherein said process comprises hydrolysis of a compound of formula (II)^{d}

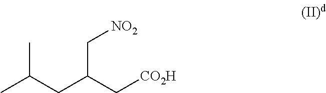

followed by hydrogenation under alkaline conditions.

It will be appreciated that the hydrolysis and hydrogenation steps may be performed in an analogous manner to those previously described hereinbefore for the first and second aspects of the invention.

Compounds of formula (II)^{d} may be prepared in accordance with Scheme 3:

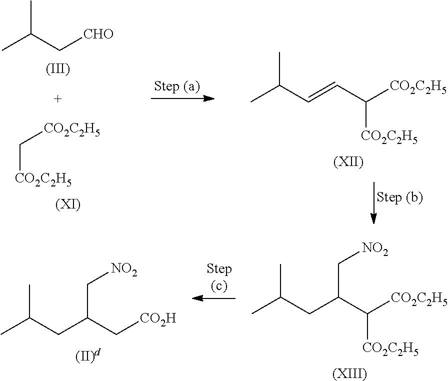

Step (a) typically comprises reaction of diethyl malonate (XI) with isovaleraldehyde (III) by adding diethyl malonate to isovaleraldehye at room temeparature. The reaction mixture was then cooled to 0-5° C. and pyridine and a catalytic amount of piperidine was then added to it. The reaction mixture was allowed to warm to room temperature and acetic acid and n-hexane were added. The reaction mixture was heated to reflux with azeotropic removal of water with a Dean-Stark apparatus. After overnight reflux, the reaction mixture was brought to room temperature. Water and n-hexane were added to the mixture. The organic layer was washed with cold dilute hydrochloric acid, water, saturated sodium bicarbonate solution, water and brine, dried over sodium sulfate, and concentrated under reduced pressure to give compound (XII) as an oil.

Step (b) typically comprises mixing the diester compound of formula (XII) with nitromethane at at 0° C., adding the DBU dropwise under an atmosphere of nitrogen. The reaction mixture is allowed to warm at room temperature. After 1 hr at room temperature, the reaction mixture is cooled to 5-10° C. and water is added to quench the reaction. The reaction mixture is then heated with n-hexane. The organic layer is washed with 10% aqueous hydrochloric acid, water and brine, dried over sodium sulfate, and concentrated under reduced pressure to give the nitro compound of formula (XIII) as an oil, which is carried over to the next step without further purification.

Step (c) typically comprises adding an aqueous solution of hydrobromic acid (47% in water) to the compound of formula (XIII) and heating the reaction mixture to reflux. After 48 hr at 110-120° C., The reaction mixture is cooled to room temperature and then poured into crushed ice. The reaction mixture is extracted with tert-butyl methyl ether. The organic layer is then washed with water, brine dried over sodium sulfate and concentrated under reduced pressure to give the nitro acid compound of formula (II)^{d} as a solid.

(±) Pregabalin may be prepared from the compound of formula (II)^{d} by adding a cold aqueous solution of sodium hydroxide or potassium hydroxide to a solution of the crude nitro compound of formula (II)^{d} in water at 0-5° C. After 1 hr at 0-5° C., the reaction mixture is allowed to attain room temperature. Methanol and ethanol were then added and, reaction mixture was then transferred to an autoclave. A slurry of Raney nickel in methanol is added to the reaction mixture. The mixture is stirred at room temperature at a hydrogen pressure of 50 psi until uptake of hydrogen is stopped. On completion of the reduction as indicated by TLC, the reaction mixture is concentrated under reduced condition to remove methanol and ethanol and then acidified with acetic acid. The precipitated material is filtered and crystallized from isopropyl alcohol-water mixture to give a racemic pregabalin, which is resolved with (S)-mandelic acid following the procedure of Marvin S. Hoekstra et al., Organic Process Research and Development, 1997, 1, 26—to give pregabalin.

It will be appreciated that certain intermediates used in the present invention are novel and therefore also constitute separate aspects of the invention. Thus, according to a further aspect of the invention there is provided an intermediate of formula (X):

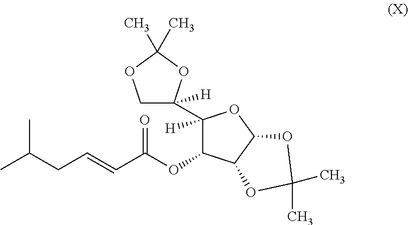

According to a further aspect of the invention there is provided an intermediate of formula (II)b:

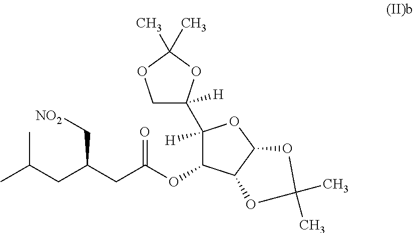

According to a further aspect of the invention there is provided an intermediate of formula (II)c:

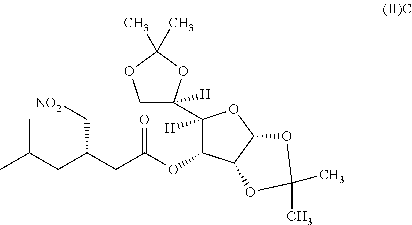

According to a further aspect of the invention there is provided an intermediate as defined hereinbefore for use in the preparation of pregabalin.

The invention will now be illustrated by the following non-limiting examples:

### EXAMPLE 1

### Ethyl 5-methylhex-2-enoate (E1)

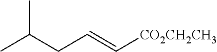

Monoethyl malonate (429 g, 3.25 mol) was added to a cold solution of isovaleraldehyde (280 g, 3.25 mol) at 0-5° C. Pyridine (22 ml) and piperidine (2 ml) were then added to the reaction mixture at 0-5° C., and the reaction mixture stirred overnight at room temperature. Thin Layer Chromatography (TLC) (n-hexane:ethyl acetate 7:3) of the reaction mixture indicated the presence of starting materials. The reaction mixture was then heated to 80° C. After 2 h at 80° C., the reaction was quenched by adding cold dilute hydrochloric acid and the product was extracted into n-hexane.

The organic layer was washed successively with cold dilute hydrochloric acid, water and brine, dried over sodium sulphate and concentrated under reduced pressure to give the ester as an oil. The crude material was then distilled under vacuum to give the ester (E1) as a colourless oil (470 g, 92%).

### EXAMPLE 2

### Ethyl (±)-3-nitromethyl-5-methylhexanoate (E2)

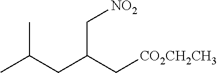

Nitromethane (257.2 g, 4.218 mol) was added to ethyl 5-methylhex-2-enoate (470 g, 3.013 mol) (which may be prepared as described in E1) at room temperature. The reaction mixture was cooled to 0-5° C., and DBU (366.8 g, 2.41 mol) was added to the mixture at 0-5° C. under an atmosphere of nitrogen. The reaction mixture was allowed to warm to room temperature, and then it was stirred for 6 h under an atmosphere of nitrogen.

After completion of reaction as indicated by TLC, the reaction was quenched by adding water (250 ml) at 0-5° C. The reaction mixture was extracted with dichloromethane. The organic layer was washed successively with water, cold dilute hydrochloric acid, water and with brine, and dried over sodium sulphate. It was then concentrated under reduced pressure to give the nitro compound (E2) as an oil. The crude material was purified by distillation to give the nitro compound (409 g, 62%) as described in R. Andruszkiewicz, R. B. Silverman, Synthesis 1989, 953-955.

### EXAMPLE 3

### (±)-3-Aminomethyl-5-methylhexanoic acid (E3)

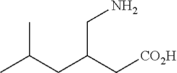

Ethyl (±)-3-nitromethyl-5-methylhexanoate (218 g, 1.005 mol) (which may be prepared as described in E2) was cooled to 10-15° C. and a cold solution potassium hydroxide (169 g dissolved in 262 ml water) was added slowly to it over a period of 45 minutes. During the addition, the temperature of the reaction mixture was not allowed to rise above 0-5° C. After the completion of addition, the reaction mixture was allowed to warm to room temperature and stirred at this temperature for 3-4 h when the reaction was completed, as indicated by TLC of the reaction mixture.

Methanol (50 ml) and ethanol (50 ml) were then added to the reaction mixture to make it homogenous. Raney nickel (40 g, 50% wet in water) was added to it and the reaction mixture was hydrogenated under a pressure of hydrogen (50 psi) at room temperature until the uptake of hydrogen stopped (20-24 h). The Raney nickel catalyst was filtered off, and the reaction mixture was concentrated under reduced pressure to a volume of about 435 ml.

The reaction mixture was then cooled to 0-3° C. and was acidified by adding dropwise cold acetic acid (180 ml) until the pH of the solution became 7. During the entire addition, the temperature of the reaction mixture was maintained at 0-3° C. The racemic pregabalin title compound (E3) (85 g, 53%) precipitated from the solution; it was filtered, washed with cold dichloromethane and resolved with (S)-mandelic acid following the procedure of Marvin S. Hoekstra et. al., Organic Process Research and Development, 1997, 1, 26-38 to give pregabalin.

The reaction was also conducted using sodium hydroxide in place of potassium hydroxide to obtain racemic pregablin.

### EXAMPLE 4

### 5-Methylhex-2-enoic acid (E4)

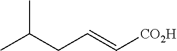

Pyridine (150 ml) was added to isovaleraldehyde (150 g, 1.741 mol) at 0-5° C. Malonic acid (290 g, 2.786 mol) was then added in batches to the reaction mixture at 0-5° C. Piperidine (10 ml) was then added to the reaction mixture at 0-5° C. The reaction mixture was allowed to warm to room temperature and stirred at this temperature for 12 h. The reactions mixture was then maintained at 80° C. for 1.5 h, when TLC indicated completion of the reaction.

The reaction was quenched by adding dilute hydrochloric acid. The aqueous layer that separated was extracted with dichloromethane (3×100 ml) and combined with the separated organic layer. The combined organic layers were washed with water (3×100 ml), cold dilute dilute hydrochloric acid, water and brine, and concentrated under reduced pressure to give the acid (E4) as an oil (200 g, 89%).

### EXAMPLE 5

### 5-Methylhex-2-enoyl chloride (E5)

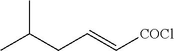

Thionyl chloride (76.7 ml, 1.05 mol) was added dropwise to 5-methylhex-2-enoic acid (103 g, 0.805 mol, which may be prepared as described in E4) at 0-5° C. and the reaction mixture was stirred at this temperature for 10 minutes. The reaction mixture was refluxed for 3 h at 80° C., and then allowed to cool to room temperature.

The excess of thionyl chloride was distilled off from the reaction mixture. Dichloromethane (50 ml) was then added to the crude reaction mixture and distilled off to remove the last traces of thionyl chloride. The crude reaction mixture was then distilled under reduced pressure to give the acid chloride (E5) (92 g, 78%) as a colourless liquid.

### EXAMPLE 6

### 1,2:5,6-Di-O-isopropylidene-α-D-glucofuranos-3-yl 5-methylhex-2-enoate (E6)

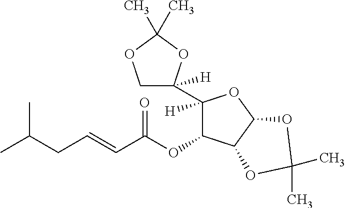

Pyridine (72.9 g, 922 mmol) was added to a stirred, cold solution of diacetone glucose (80 g, 307.7 mmol) at 0-5° C. After 15 minutes at 0-5° C., 5-methylhex-2-enoyl chloride (59.07 g, 404.6 mmol) (which may be prepared as described in E5) was added dropwise and the reaction mixture was stirred at this temperature for another 15 minutes. A catalytic amount of N,N-dimethylaminopyridine (DMAP) (0.5 g) was then added, the reaction mixture was allowed to warm to room temperature and stirred for 4 h at this temperature when the TLC indicated completion of reaction.

The dichloromethane was evaporated off under reduced pressure and the residue was diluted with water. The mixture was extracted with n-hexane (4×60 ml). The organic layer was washed successively with water (7×20 ml), dilute hydrochloric acid (3×20 ml), water (3×20 ml) and with brine (2×20 ml), and concentrated under reduced pressure to give the ester (E6) (95 g, 63%).

### EXAMPLE 7

### 1,2:5,6-Di-O-isopropylidene-α-D-glucofuranos-3-yl 3-nitromethyl-5-methylhexanoate (E7)

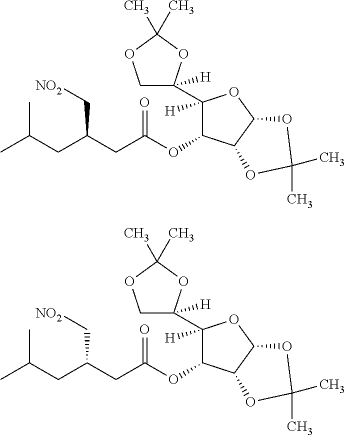

Nitromethane (89.9 g, 1.474 mol) was added to 1,2:5,6-di-O-isopropylidene-α-D-glucofuranos-3-yl 5-methylhex-2-enoate (105 g, 0.284 mol) (which may be prepared as described in E6) and the reaction mixture was cooled to −20° C. DBU (62.7 g, 0.412 mmol) was then added dropwise at −20° C. and the reaction mixture was maintained in the temperature range −20° C.-25° C. for 4 h. TLC analysis of the reaction mixture indicated completion of the reaction.

The reaction was quenched with water and extracted with n-hexane (5×50 ml). The organic layer was washed successively with water (7×20 ml), dilute hydrochloric acid (3×20 ml), water (3×20 ml) and with brine (2×20 ml), dried over sodium sulphate, and concentrated under reduced pressure to give a diasteromeric mixture of the title compounds (E7) (79 g, 65%).

### EXAMPLE 8

### (+) and (−)-3-Aminomethyl-5-methylhexanoic acid (E8)

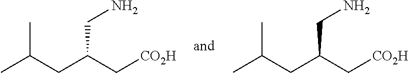

The diastereoisomeric mixture of 1,2:5,6-Di-O-isoproPylidene-α-D-qlucofuranos-3-yl 3-nitromethyl-5-methylhexanoate (which may be prepared as described in E7) was hydrolysed with aqueous potassium hydroxide and was extracted with dichloromethane. The organic layer was evaporated after the usual work-up to recover the chiral auxiliary. The aqueous layer was subjected to Raney nickel-catalysed reduction under hydrogen pressure in a similar manner as detailed in Example 3 to give the title compound (E8) as enantiomerically enriched pregabalin. The enantiomeric excess was not determined.

### EXAMPLE 9

### Ethyl (±) 3-nitromethyl-5-methylhexanoic Acid (E9)

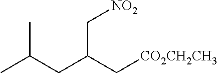

Nitromethane (195.64 g) was added to the compound of Example 1 (500 g) at room temperature. The reaction mixture was cooled to 0-5° C., and N,N-tetramethylguanidine (369.16 g) was added to the mixture at 0-5° C. under an atmosphere of nitrogen. Tie reaction mixture was allowed to warm to room temperature and then it was stirred at this temperature for 24-30 h under an atmosphere of nitrogen.

After completion of the reaction as indicated by TLC, the reaction mixture was subjected to distillation under reduced pressure to recover N,N-tetramethylguanidine. It distilled out from the mixture at a bath temperature range of 61-71° C. at 1 mm of Hg and weighed 277.5 g and it was recycled in subsequent reactions. The residue after the removal of N,N-tetramethylguanidine contained primarily the title compound (GC purity of the residue: 80%). The crude material was carried over to the following step without further purification.

### EXAMPLE 10

### (±)-3-Aminomethyl-5-methylhexanoic-acid (E10)

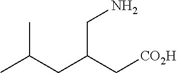

The compound of Example 9 (523.5 g) was cooled to 10-15° C. and a cold solution of potassium hydroxide (410.91 g dissolved in 623 ml water) was added slowly to it over a period of 45 minutes. During the addition, the temperature of the reaction mixture was not allowed to rise above 0-5° C. After the completion of addition, the reaction mixture was allowed to warm to room temperature and stirred at this temperature for 3-4 h when the reaction was completed, as indicated by TLC of the reaction mixture.

Methanol (119.5 ml) and ethanol (119.5 ml) were then added to the reaction mixture to make it homogenous. Raney nickel (52.3 g, 50% wet in water) was added to it and the reaction mixture was hydrogenated under a pressure of hydrogen (50 psi) at room temperature until the uptake of hydrogen stopped (20-24 h). The Raney nickel catalyst was filtered off, and the reaction mixture was concentrated under reduced pressure to a volume of about 435 ml.

The reaction mixture was then cooled to 0-3° C. and was acidified by adding dropwise cold acetic acid (180 ml) until the pH of the solution became 7. During the entire addition, the temperature of the reaction mixture was maintained at 0-3° C.

### EXAMPLE 11

### (−)-3-Aminomethyl-5-methylhexanoic acid (E11)

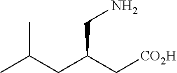

The racemic pregabalin of Example 10 was filtered, washed with cold dibhloromethane and resolved with (S)-mandelic acid using isopropanol-water (97:3) solvent system following the procedure of Marvin S. Hoekstra et. al., Organic Process Research and Development, 1997, 1, 26, to obtain the first batch of pregabalin. Another batch of pregabalin was obtained from the filtrates of the first resolution following the procedure as detailed under:

The filtrates of the first resolution were combined and evaporated completely under reduced pressure to recover isopropyl alcohol, which was subsequently reused without any loss of either the purity or the yield of the final product, Pregabalin. The residue thus obtained was acidified with concentrated hydrochloric acid triggering the precipitation of the first batch of S(+)-Mandelic acid. The aqueous solution was then extracted with ethyl acetate. The organic layer was washed with water, brine, dried over sodium sulphate and evaporated under reduced pressure to give another batch of S(+)-Mandelic acid as a white solid which was combined with the former and recycled in subsequent batches without any loss of either the purity or the yield of the final product, Pregabalin. The aqueous layer containing the amino acid enriched with the undesired isomer was evaporated under reduced pressure to a thick oil and basified with triethyl amine at −5° C. to 0° C. Chloroform was then added to it at −5° C. to 0° C. and the mixture was maintained at this temperature for 1-2 h. The precipitated solid was filtered, washed with cold chloroform and air-dried overnight to give the amino acid enriched with the undesired isomer which was resolved with R(−) Mandelic acid repeating the procedure of Marvin S. Hoekstra et. al., Organic Process Research and Development, 1997, 1, 26; to obtain the undesired isomer of pregabalin. The filtrates of this resolution was combined and evaporated under reduced pressure to recover and recycle isopropyl alcohol. The residue thus obtained was subjected to the process of recovery of R(−) Mandelic acid and the racemic Pregabalin following the protocol mentioned in the preceding paragraph. R(−) Mandelic acid was recycled in subsequent batches without any loss of the yield or the purity of the undesired isomer of Pregabalin. The recovered racemic Pregabalin thus obtained was again resolved, with S(+)-Mandelic acid repeating the Marvin S. Hoekstra et. al., Organic Process Research and Development, 1997, 1, 26, to obtain a second batch of Pregabalin.

The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to a person skilled in the art are intended to be included within the scope of the invention as defined by the accompanying claims.

We claim:

1. A process for preparing a compound of formula (I): 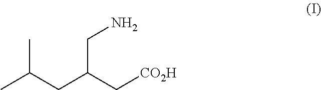
wherein said process comprises hydrogenation of a compound of formula (II) 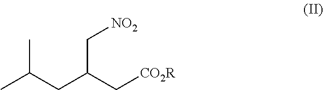
under alkaline conditions, wherein R represents hydrogen or a labile group capable of being converted to hydrogen.

2. A process as defined in claim 1, wherein R represents a labile group capable of being converted to hydrogen.

3. A process for preparing a compound of formula (I): 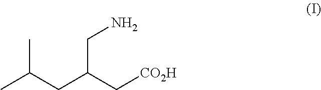
wherein said process comprises hydrolysis of a compound of formula (II) 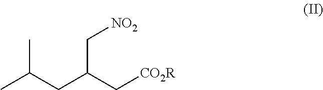
followed by hydrogenation under alkaline conditions, wherein R represents a labile group capable of being converted to hydrogen.

4. A process as defined in any preceding claims, wherein R represents C₁₋₆ alkyl, such as ethyl.

5. A process as defined in any preceding claims, wherein R represents a compound of formula R^{a}: 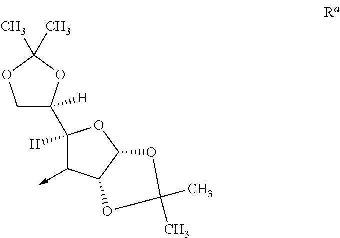
wherein [symbol] represents the point of attachment of R to the O atom of the CO₂ group.

6. A process as defined in claim 1, wherein R represents hydrogen.

7. A process for preparing a compound of formula (I): 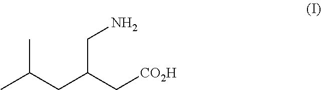
wherein said process comprises hydrolysis of a compound of formula (II)^{d} 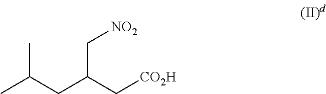
followed by hydrogenation under alkaline conditions.

8. A process as defined in any preceding claims, wherein said alkaline conditions are created by addition of a suitable base, such as potassium hydroxide.

9. A process as defined in any preceding claims, wherein said hydrogenation is conducted in the presence of a suitable catalyst, such as a catalyst comprising a metal selected from nickel, platinum, palladium, rhodium and ruthenium.

10. A process as defined in claim 9, wherein said catalyst comprises a metal selected from nickel, such as Raney nickel or Urushibara nickel, in particular, Raney nickel.

11. A process as defined in any preceding claims, wherein the hydrogenation reaction of the invention comprises hydrogenation at a pressure of between 100 and 500 kPa, such as 345 kPa.

12. A process as defined in any preceding claims, wherein the hydrolysis reaction comprises the addition of potassium hydroxide, such as potassium hydroxide cooled to 10-15° C.

13. A process as defined in claim 10, wherein said potassium hydroxide is added at a temperature of between 0 and 5° C.

14. A process as defined in any preceding claims, wherein the hydrolysis reaction is conducted at room temperature.

15. A process as defined in any preceding claims which additionally comprises the step of preparing a resolved form of the compound of formula (I): 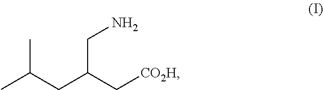
such as a compound of formula (I)^{b} 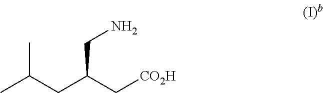
which comprises resolution of the racemic compound of formula (I) by S(+)-mandelic acid.

16. A process for preparing a resolved form of the compound of formula (I): 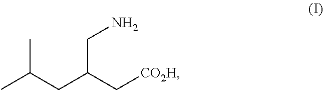
such as a compound of formula (I)^{b} 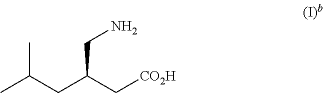
which comprises resolution of the racemic compound of formula (I) by S(+)-mandelic acid.

17. A purified compound of formula (I)^{b} 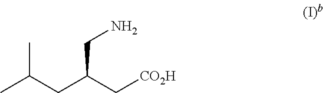
obtainable by the resolution process as defined in claim 15 or claim 16.

18. An intermediate of formula (X):

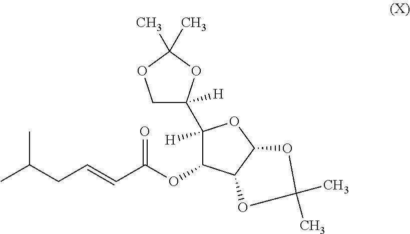

19. An intermediate of formula (II)b:

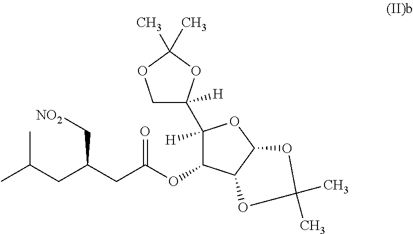

20. An intermediate of formula (II)c:

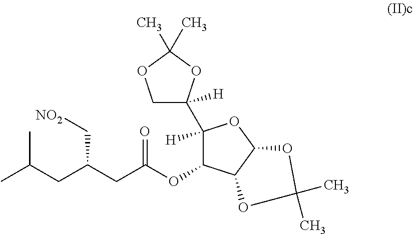

21. An intermediate as defined in any of claims 18 to 20 for use in the preparation of pregabalin.
